(19) United States
(12) Patent Application Publication
(10) Pub. No.: US 2019/0142386 A1
(43) Pub. Date: May 16, 2019

(54) SYSTEMS AND METHODS FOR MULTI-RESOLUTION DISCRIMINANT ANALYSIS FOR ULTRASOUND IMAGING

(71) Applicant: EDAN INSTRUMENTS, INC., Shenzhen (CN)

(72) Inventors: Seshadri Srinivasan, Sunnyvale, CA (US); Ruiying Zhang, Shenzhen (CN)

(73) Assignee: EDAN INSTRUMENTS, INC., Shenzhen (CN)

(21) Appl. No.: 16/189,920

(22) Filed: Nov. 13, 2018

Related U.S. Application Data
(60) Provisional application No. 62/586,004, filed on Nov. 14, 2017.

(51) Int. Cl. A61B 8/08; G06T 7/00; G06K 9/00; A61B 8/00; A61B 8/06

(52) U.S. Cl. CPC A61B 8/5207; G06T 7/0012; G06T 2207/10132; A61B 8/46; A61B 8/06; G06K 9/0055

(57) ABSTRACT

An ultrasound system includes an ultrasound transducer, a processing circuit, and an output device. The ultrasound transducer detects ultrasound information and outputs the ultrasound information as ultrasound data samples. The processing circuit receives ultrasound data samples from the ultrasound transducer, calculates a plurality of first spectra for a first subset of the received ultrasound data samples, generates a threshold based on the plurality of first spectra, categorizes first spectra greater than the threshold as signal data, otherwise as noise data, processes the signal data using a first signal processing parameter and the noise data using a second signal processing parameter different from the first signal processing parameter, and combines the processed signal data and noise data into an ultrasound output. The output device is configured to output the ultrasound output as at least one of an ultrasound image or ultrasound audio.

### CROSS-REFERENCE TO RELATED APPLICATION

This application claims priority from U.S. Provisional Patent Application No. 62/586,004, filed Nov. 14, 2017. The contents of this application is incorporated herein by reference in its entirety.

### TECHNICAL FIELD

The present disclosure generally relates to ultrasound systems. In some implementations, the present disclosure relates to ultrasound systems that perform multi-resolution discriminant analysis for ultrasound imaging.

### BACKGROUND

Ultrasound systems can be used to detect information regarding a patient, including information regarding blood flow in a patient, in order to display such information to a medical professional or other user so that the user can make medical decisions based on the information. For example, an ultrasound transducer can transmit ultrasound waves into a body of the patient and detect return waves that may have been modified by blood flow and vascular structures of the body of the patient, and a computer can communicate with the ultrasound transducer to receive ultrasound information from the ultrasound transducer and display spectra and/or images using the ultrasound information.

However, various factors involved in the process of detecting and displaying ultrasound information may make it difficult to distinguish vascular features from blood flow, which can reduce the signal to noise ratio of the information ultimately provided to the user. Existing techniques often perform poorly in low signal-to-noise ratio environments, including for both pulse wave and continuous wave operation. In addition, existing techniques used to improve the signal to noise ratio may distort the ultrasound information when applied to both blood flow and vascular features. As such, it may be difficult to display such information in an accurate and easily understood manner and thus difficult for the user to make medical decisions based on the information.

### SUMMARY

One embodiment relates to a system. The system includes an ultrasound transducer, a processing circuit, and an output device. The ultrasound transducer is configured to detect ultrasound information regarding a patient and output the ultrasound information as ultrasound data samples. The processing circuit is configured to receive a plurality of ultrasound data samples from the ultrasound transducer, calculate a plurality of first spectra for a first subset of the received ultrasound data samples, generate a threshold based on the plurality of first spectra, compare each of the plurality of first spectra to the threshold, categorize first spectra greater than the threshold as signal data, categorize first spectra less than or equal to the threshold as noise data, process the signal data using a first signal processing parameter, process the noise data using a second signal processing parameter different from the first signal processing parameter, and combine the processed signal data and noise data into an ultrasound output. The output device includes at least one of a display configured to display the ultrasound output as an ultrasound image or an audio output device configured to output the ultrasound output as ultrasound audio.

Another embodiment relates to a portable ultrasound device. The portable ultrasound device includes a processing circuit configured to calculate a plurality of first spectra for a first subset of the received ultrasound data samples, generate a threshold based on the plurality of first spectra, compare each of the plurality of first spectra to the threshold, categorize first spectra greater than the threshold as signal data, categorize first spectra less than or equal to the threshold as noise data, process the signal data using a first signal processing parameter, process the noise data using a second signal processing parameter different from the first signal processing parameter, and combine the processed signal data and noise data into an ultrasound output.

Another embodiment relates to a method. The method includes receiving a plurality of ultrasound data samples associated with ultrasound information regarding a patient, calculating a plurality of first spectra for a first subset of the received ultrasound data samples, generating a threshold based on the plurality of first spectra, comparing each of the plurality of first spectra to the threshold, categorizing first spectra greater than the threshold as signal data, categorizing first spectra less than or equal to the threshold as noise data, processing the signal data using a first signal processing parameter, processing the noise data using a second signal processing parameter different from the first signal processing parameter, combining the processing signal data and noise data into an ultrasound output, and at least one of displaying the ultrasound output as an ultrasound image or outputting the ultrasound output as ultrasound audio.

### BRIEF DESCRIPTION OF THE DRAWINGS

FIG. 1A is a perspective view of an ultrasound system according to an illustrative embodiment.

FIG. 1B is a perspective view of components of an ultrasound system according to an illustrative embodiment.

FIG. 2 is a block diagram illustrating components of an ultrasound system according to an illustrative embodiment.

FIG. 3 is a block diagram illustrating components of a processing circuit of an ultrasound system according to an illustrative embodiment.

FIG. 4 is a flow chart of a method for multi-resolution discriminant analysis for ultrasound data, according an embodiment of the present disclosure.

### DETAILED DESCRIPTION

Before turning to the Figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

Referring to the Figures generally, a system can include an ultrasound transducer, a processing circuit, and an output device. The ultrasound transducer is configured to detect ultrasound information regarding a patient and output the ultrasound information as ultrasound data samples. The processing circuit is configured to receive a plurality of ultrasound data samples from the ultrasound transducer, calculate a plurality of first spectra for a first subset of the received ultrasound data samples, generate a threshold based on the plurality of first spectra, compare each of the plurality of first spectra to the threshold, categorize first spectra greater than the threshold as signal data, categorize first spectra less than or equal to the threshold as noise data, process the signal data using a first signal processing parameter, process the noise data using a second signal processing parameter different from the first signal processing parameter, and combine the processed signal data and noise data into an ultrasound output. The output device includes at least one of a display configured to display the ultrasound output as an ultrasound image or an audio output device configured to output the ultrasound output as ultrasound audio.

By using the threshold to categorize ultrasound data as signal data or noise data, and then processing the signal data and noise data using different signal processing parameters, systems and methods as described herein can improve the display (or audio output) of ultrasound information, such as by increasing the signal-to-noise ratio, improving spectral resolution, more clearly identifying anatomical features, more clearly distinguishing blood flow from vessel walls, and otherwise more accurately representing the underlying anatomy being representing using ultrasound devices.

### A. Ultrasound System

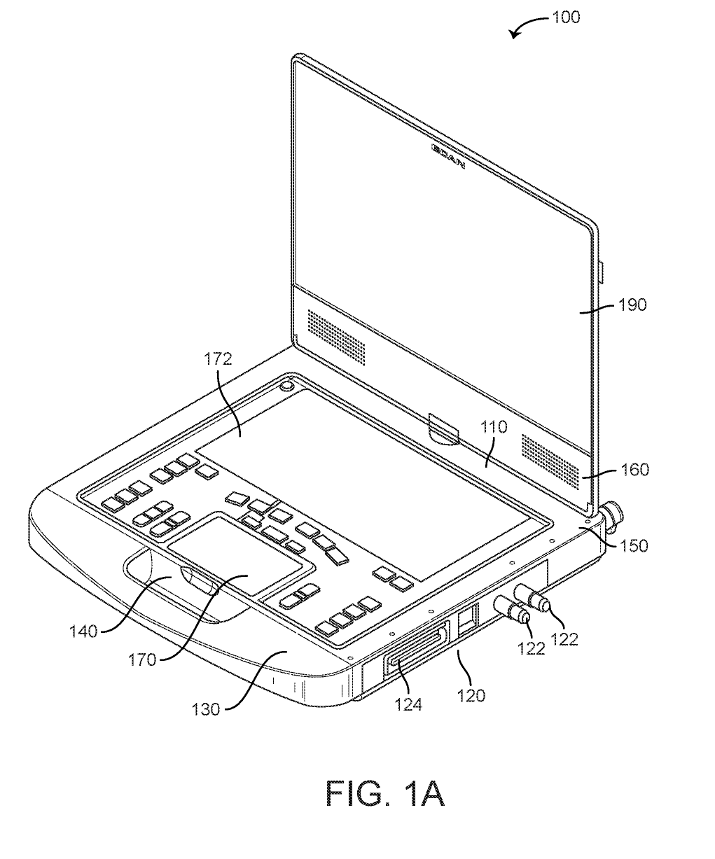

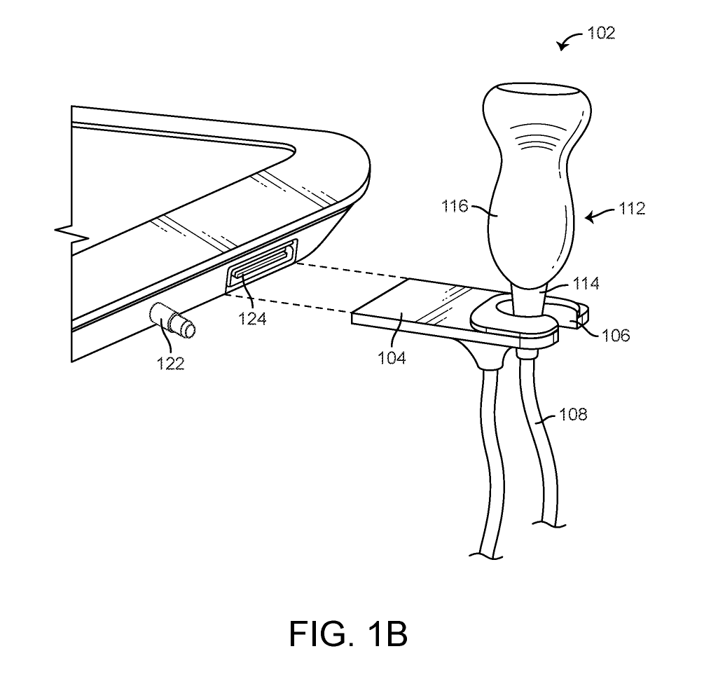

Referring now to FIG. 1A, an embodiment of portable ultrasound system 100 is illustrated. Portable ultrasound system 100 may include display support system 110 for increasing the durability of the display system. Portable ultrasound system 100 may further include locking lever system 120 for securing ultrasound probes and/or transducers. Some embodiments of portable ultrasound system 100 include ergonomic handle system 130 for increasing portability and usability. Further embodiments include status indicator system 140 which displays, to a user, information relevant to portable ultrasound system 100. Portable ultrasound system 100 may further include features such as an easy to operate and customizable user interface, adjustable feet, a backup battery, modular construction, cooling systems, etc.

Still referring to FIG. 1A, main housing 150 houses components of portable ultrasound system 100. In some embodiments, the components housed within main housing 150 include locking lever system 120, ergonomic handle system 130, and status indicator system 140. Main housing 150 may also be configured to support electronics modules which may be replaced and/or upgraded due to the modular construction of portable ultrasound system 100. In some embodiments, portable ultrasound system 100 includes display housing 160. Display housing 160 may include display support system 110. In some embodiments, portable ultrasound system 100 includes touchpad 170 for receiving user inputs and displaying information, touchscreen 172 for receiving user inputs and displaying information, and main screen 190 for displaying information. While FIG. 1A illustrates the portable ultrasound system 100 as being implemented with hinged main housing 150 and multiple display screens, touchpads, and/or touchscreens, it will be appreciated that the portable ultrasound system 100 may be implemented using various portable electronic devices, including tablets or other portable electronic devices having a single touchscreen.

Referring now to FIG. 1B, ultrasound transducer assembly 102 is shown. According to an exemplary embodiment, ultrasound transducer assembly 102 includes a connection assembly to pin (122) or socket (124) type ultrasound interface, shown as ultrasound interface connector 104, coupled to cable 108. Cable 108 may be coupled to a transducer probe 112. While FIG. 1B shows only one transducer assembly 102, more transducer assemblies may be coupled to the ultrasound system 100 based on the quantity of pin (122) or socket (124) type ultrasound interfaces.

Ultrasound interface connector 104 is movable between a removed position with respect to pin (122) or socket (124) type ultrasound interface, in which ultrasound interface connector 104 is not received by pin (122) or socket (124) type ultrasound interface, a partially connected position, in which ultrasound interface connector 104 is partially received by pin (122) or socket (124) type ultrasound interface, and a fully engaged position, in which ultrasound interface connector 104 is fully received by pin (122) or socket (124) type ultrasound interface in a manner that electrically couples transducer probe 112 to ultrasound system 100. In an exemplary embodiment, pin (122) or socket (124) type ultrasound interface may include a sensor or switch that detects the presence of the ultrasound interface connector 104.

In various exemplary embodiments contained herein, the ultrasound interface connector 104 may house passive or active electronic circuits for affecting the performance of the connected transducers. For example, in some embodiments the transducer assembly 102 may include filtering circuitry, processing circuitry, amplifiers, transformers, capacitors, batteries, failsafe circuits, or other electronics which may customize or facilitate the performance of the transducer and/or the overall ultrasound machine. In an exemplary embodiment, ultrasound interface connector 104 may include a bracket 106, where the transducer probe 112 may be stored when not in use.

Transducer probe 112 transmits and receives ultrasound signals that interact with the patient during the diagnostic ultrasound examination. The transducer probe 112 includes a first end 114 and a second end 116. The first end 114 of the transducer probe 112 may be coupled to cable 108. The first end 114 of the transducer probe 112 may vary in shape to properly facilitate the cable 108 and the second end 116. The second end 116 of the transducer probe 112 may vary in shape and size to facilitate the conduction of different types of ultrasound examinations. These first end 114 and second end 116 of transducer probe 112 variations may allow for better examination methods (e.g., contact, position, location, etc.).

A user (e.g., a sonographer, an ultrasound technologist, etc.) may remove a transducer probe 112 from a bracket 106 located on ultrasound interface connector 104, position transducer probe 112, and interact with main screen 190 to conduct the diagnostic ultrasound examination. Conducting the diagnostic ultrasound examination may include pressing transducer probe 112 against the patient's body or placing a variation of transducer probe 112 into the patient. The ultrasound spectrum or image acquired may be viewed on the main screen 190.

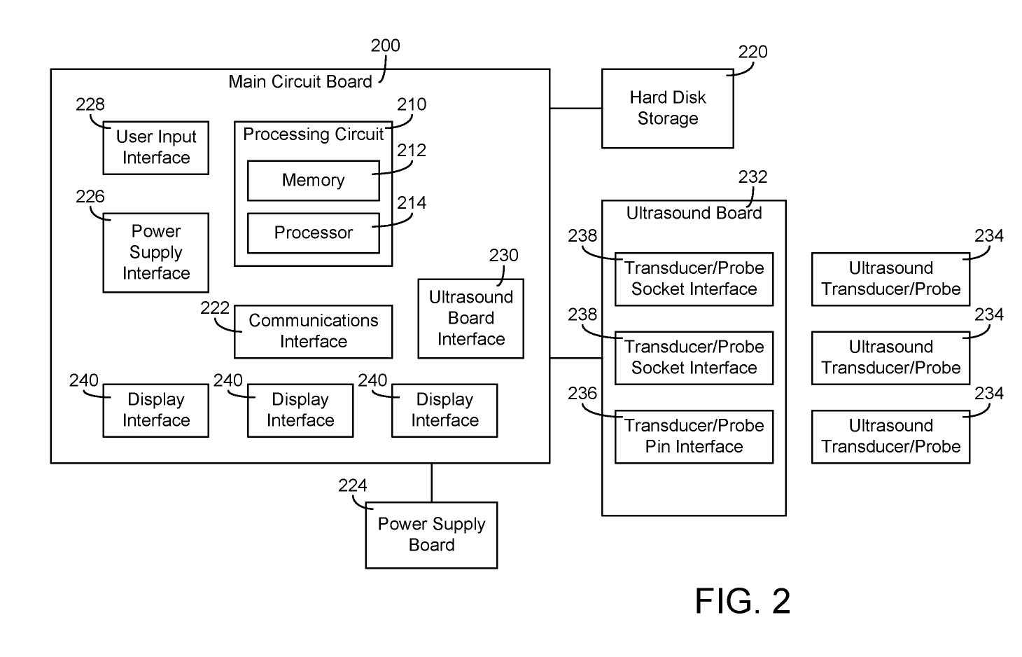

Referring to FIG. 2, a block diagram shows internal components of one embodiment of portable ultrasound system 100. Portable ultrasound system 100 includes main circuit board 200. Main circuit board 200 carries out computing tasks to support the functions of portable ultrasound system 100 and provides connection and communication between various components of portable ultrasound system 100. In some embodiments, main circuit board 200 is configured so as to be a replaceable and/or upgradable module.

To perform computational, control, and/or communication tasks, main circuit board 200 includes processing circuit 210. Processing circuit 210 is configured to perform general processing and to perform processing and computational tasks associated with specific functions of portable ultrasound system 100. For example, processing circuit 210 may perform calculations and/or operations related to producing a spectrum and/or an image from signals and/or data provided by ultrasound equipment, running an operating system for portable ultrasound system 100, receiving user inputs, etc. Processing circuit 210 may include memory 212 and processor 214 for use in processing tasks. For example, processing circuit 210 may perform calculations and/or operations.

Processor 214 may be, or may include, one or more microprocessors, application specific integrated circuits (ASICs), circuits containing one or more processing components, a group of distributed processing components, circuitry for supporting a microprocessor, or other hardware configured for processing. Processor 214 is configured to execute computer code. The computer code may be stored in memory 212 to complete and facilitate the activities described herein with respect to portable ultrasound system 100. In other embodiments, the computer code may be retrieved and provided to processor 214 from hard disk storage 220 or communications interface 222 (e.g., the computer code may be provided from a source external to main circuit board 200).

Memory 212 may be any volatile or non-volatile computer-readable storage medium capable of storing data or computer code relating to the activities described herein. For example, memory 212 may include modules which are computer code modules (e.g., executable code, object code, source code, script code, machine code, etc.) configured for execution by processor 214. Memory 212 may include computer code engines or circuits that can be similar to the computer code modules configured for execution by processor 214. Memory 212 may include computer executable code related to functions including ultrasound imagining, battery management, handling user inputs, displaying data, transmitting and receiving data using a wireless communication device, etc. In some embodiments, processing circuit 210 may represent a collection of multiple processing devices (e.g., multiple processors, etc.). In such cases, processor 214 represents the collective processors of the devices and memory 212 represents the collective storage devices of the devices. When executed by processor 214, processing circuit 210 is configured to complete the activities described herein as associated with portable ultrasound system 100, such as for generating ultrasound spectra, images, and/or audio (e.g., for display by touchscreen 172 and/or display 190) based on multi-resolution discriminant analysis.

Hard disk storage 220 may be a part of memory 212 and/or used for non-volatile long term storage in portable ultrasound system 100. Hard disk storage 220 may store local files, temporary files, ultrasound spectra and/or images, patient data, an operating system, executable code, and any other data for supporting the activities of portable ultrasound device 100 described herein. In some embodiments, hard disk storage 220 is embedded on main circuit board 200. In other embodiments, hard disk storage 220 is located remote from main circuit board 200 and coupled thereto to allow for the transfer of data, electrical power, and/or control signals. Hard disk storage 220 may be an optical drive, magnetic drive, a solid state hard drive, flash memory, etc.

In some embodiments, main circuit board 200 includes communications interface 222. Communications interface 222 may include connections which enable communication between components of main circuit board 200 and communications hardware. For example, communications interface 222 may provide a connection between main circuit board 200 and a network device (e.g., a network card, a wireless transmitter/receiver, etc.). In further embodiments, communications interface 222 may include additional circuitry to support the functionality of attached communications hardware or to facilitate the transfer of data between communications hardware and main circuit board 200. In other embodiments, communications interface 222 may be a system on a chip (SOC) or other integrated system which allows for transmission of data and reception of data. In such a case, communications interface 222 may be coupled directly to main circuit board 200 as either a removable package or embedded package.

Some embodiments of portable ultrasound system 100 include power supply board 224. Power supply board 224 includes components and circuitry for delivering power to components and devices within and/or attached to portable ultrasound system 100. In some embodiments, power supply board 224 includes components for alternating current and direct current conversion, for transforming voltage, for delivering a steady power supply, etc. These components may include transformers, capacitors, modulators, etc. to perform the above functions. In further embodiments, power supply board 224 includes circuitry for determining the available power of a battery power source. In other embodiments, power supply board 224 may receive information regarding the available power of a battery power source from circuitry located remote from power supply board 224. For example, this circuitry may be included within a battery. In some embodiments, power supply board 224 includes circuitry for switching between power sources. For example, power supply board 224 may draw power from a backup battery while a main battery is switched. In further embodiments, power supply board 224 includes circuitry to operate as an uninterruptable power supply in conjunction with a backup battery. Power supply board 224 also includes a connection to main circuit board 200. This connection may allow power supply board 224 to send and receive information from main circuit board 200. For example, power supply board 224 may send information to main circuit board 200 allowing for the determination of remaining battery power. The connection to main circuit board 200 may also allow main circuit board 200 to send commands to power supply board 224. For example, main circuit board 200 may send a command to power supply board 224 to switch from one source of power to another (e.g., to switch to a backup battery while a main battery is switched). In some embodiments, power supply board 224 is configured to be a module. In such cases, power supply board 224 may be configured so as to be a replaceable and/or upgradable module. In some embodiments, power supply board 224 is or includes a power supply unit. The power supply unit may convert AC power to DC power for use in portable ultrasound system 100. The power supply may perform additional functions such as short circuit protection, overload protection, undervoltage protection, etc. The power supply may conform to ATX specification. In other embodiments, one or more of the above described functions may be carried out by main circuit board 200.

Main circuit board 200 may also include power supply interface 226 which facilitates the above described communication between power supply board 224 and main circuit board 200. Power supply interface 226 may include connections which enable communication between components of main circuit board 200 and power supply board 224. In further embodiments, power supply interface 226 includes additional circuitry to support the functionality of power supply board 224. For example, power supply interface 226 may include circuitry to facilitate the calculation of remaining battery power, manage switching between available power sources, etc. In other embodiments, the above described functions of power supply board 224 may be carried out by power supply interface 226. For example, power supply interface 226 may be a SOC or other integrated system. In such a case, power supply interface 226 may be coupled directly to main circuit board 200 as either a removable package or embedded package.

With continued reference to FIG. 2, some embodiments of main circuit board 200 include user input interface 228. User input interface 228 may include connections which enable communication between components of main circuit board 200 and user input device hardware. For example, user input interface 228 may provide a connection between main circuit board 200 and a capacitive touchscreen, resistive touchscreen, mouse, keyboard, buttons, and/or a controller for the proceeding. In one embodiment, user input interface 228 couples controllers for touchpad 170, touchscreen 172, and main screen 190 to main circuit board 200. In other embodiments, user input interface 228 includes controller circuitry for touchpad 170, touchscreen 172, and main screen 190. In some embodiments, main circuit board 200 includes a plurality of user input interfaces 228. For example, each user input interface 228 may be associated with a single input device (e.g., touchpad 170, touchscreen 172, a keyboard, buttons, etc.).

In further embodiments, user input interface 228 may include additional circuitry to support the functionality of attached user input hardware or to facilitate the transfer of data between user input hardware and main circuit board 200. For example, user input interface 228 may include controller circuitry so as to function as a touchscreen controller. User input interface 228 may also include circuitry for controlling haptic feedback devices associated with user input hardware. In other embodiments, user input interface 228 may be a SOC or other integrated system which allows for receiving user inputs or otherwise controlling user input hardware. In such a case, user input interface 228 may be coupled directly to main circuit board 200 as either a removable package or embedded package.

Main circuit board 200 may also include ultrasound board interface 230 which facilitates communication between ultrasound board 232 and main circuit board 200. Ultrasound board interface 230 may include connections which enable communication between components of main circuit board 200 and ultrasound board 232. In further embodiments, ultrasound board interface 230 includes additional circuitry to support the functionality of ultrasound board 232. For example, ultrasound board interface 230 may include circuitry to facilitate the calculation of parameters used in generating a spectrum and/or an image from ultrasound data provided by ultrasound board 232. In some embodiments, ultrasound board interface 230 is a SOC or other integrated system. In such a case, ultrasound board interface 230 may be coupled directly to main circuit board 200 as either a removable package or embedded package.

In other embodiments, ultrasound board interface 230 includes connections which facilitate use of a modular ultrasound board 232. Ultrasound board 232 may be a module (e.g., ultrasound module) capable of performing functions related to ultrasound imaging (e.g., multiplexing sensor signals from an ultrasound probe/transducer, controlling the frequency of ultrasonic waves produced by an ultrasound probe/transducer, etc.). The connections of ultrasound board interface 230 may facilitate replacement of ultrasound board 232 (e.g., to replace ultrasound board 232 with an upgraded board or a board for a different application). For example, ultrasound board interface 230 may include connections which assist in accurately aligning ultrasound board 232 and/or reducing the likelihood of damage to ultrasound board 232 during removal and/or attachment (e.g., by reducing the force required to connect and/or remove the board, by assisting, with a mechanical advantage, the connection and/or removal of the board, etc.).

In embodiments of portable ultrasound system 100 including ultrasound board 232, ultrasound board 232 includes components and circuitry for supporting ultrasound imaging functions of portable ultrasound system 100. In some embodiments, ultrasound board 232 includes integrated circuits, processors, and memory. Ultrasound board 232 may also include one or more transducer/probe socket interfaces 238. Transducer/probe socket interface 238 enables ultrasound transducer/probe 234 (e.g., a probe with a socket type connector) to interface with ultrasound board 232. For example, transducer/probe socket interface 238 may include circuitry and/or hardware connecting ultrasound transducer/probe 234 to ultrasound board 232 for the transfer of electrical power and/or data. Transducer/probe socket interface 238 may include hardware which locks ultrasound transducer/probe 234 into place (e.g., a slot which accepts a pin on ultrasound transducer/probe 234 when ultrasound transducer/probe 234 is rotated). In some embodiments, ultrasound board 232 includes two transducer/probe socket interfaces 238 to allow the connection of two socket type ultrasound transducers/probes 187.

In some embodiments, ultrasound board 232 also includes one or more transducer/probe pin interfaces 236. Transducer/probe pin interface 236 enables an ultrasound transducer/probe 234 with a pin type connector to interface with ultrasound board 232. Transducer/probe pin interface 236 may include circuitry and/or hardware connecting ultrasound transducer/probe 234 to ultrasound board 232 for the transfer of electrical power and/or data. Transducer/probe pin interface 236 may include hardware which locks ultrasound transducer/probe 234 into place. In some embodiments, ultrasound transducer/probe 234 is locked into place with locking lever system 120. In some embodiments, ultrasound board 232 includes more than one transducer/probe pin interfaces 236 to allow the connection of two or more pin type ultrasound transducers/probes 234. In such cases, portable ultrasound system 100 may include one or more locking lever systems 120. In further embodiments, ultrasound board 232 may include interfaces for additional types of transducer/probe connections.

With continued reference to FIG. 2, some embodiments of main circuit board 200 include display interface 240. Display interface 240 may include connections which enable communication between components of main circuit board 200 and display device hardware. For example, display interface 240 may provide a connection between main circuit board 200 and a liquid crystal display, a plasma display, a cathode ray tube display, a light emitting diode display, and/or a display controller or graphics processing unit for the proceeding or other types of display hardware. In some embodiments, the connection of display hardware to main circuit board 200 by display interface 240 allows a processor or dedicated graphics processing unit on main circuit board 200 to control and/or send data to display hardware. Display interface 240 may be configured to send display data to display device hardware in order to produce a spectrum and/or an image. In some embodiments, main circuit board 200 includes multiple display interfaces 240 for multiple display devices (e.g., three display interfaces 240 connect three displays to main circuit board 200). In other embodiments, one display interface 240 may connect and/or support multiple displays. In one embodiment, three display interfaces 240 couple touchpad 170, touchscreen 172, and main screen 190 to main circuit board 200.

In further embodiments, display interface 240 may include additional circuitry to support the functionality of attached display hardware or to facilitate the transfer of data between display hardware and main circuit board 200. For example, display interface 240 may include controller circuitry, a graphics processing unit, video display controller, etc. In some embodiments, display interface 240 may be a SOC or other integrated system which allows for displaying spectra and/or images with display hardware or otherwise controlling display hardware. Display interface 240 may be coupled directly to main circuit board 200 as either a removable package or embedded package. Processing circuit 210 in conjunction with one or more display interfaces 240 may display spectra and/or images on one or more of touchpad 170, touchscreen 172, and main screen 190.

Referring back to FIG. 1A, in some embodiments, portable ultrasound system 100 includes one or more pin type ultrasound probe interfaces 122. Pin type ultrasound interface 122 may allow an ultrasound probe to connect to an ultrasound board 232 included in ultrasound system 100. For example, an ultrasound probe connected to pin type ultrasound interface 122 may be connected to ultrasound board 232 via transducer/probe pin interface 236. In some embodiments, pin type ultrasound interface 122 allows communication between components of portable ultrasound system 100 and an ultrasound probe. For example, control signals may be provided to the ultrasound probe 112 (e.g., controlling the ultrasound emissions of the probe) and data may be received by ultrasound system 100 from the probe (e.g., imaging data).

In some embodiments, ultrasound system 100 may include locking lever system 120 for securing an ultrasound probe. For example, an ultrasound probe may be secured in pin type ultrasound probe interface 122 by locking lever system 120.

In further embodiments, ultrasound system 100 includes one or more socket type ultrasound probe interfaces 124. Socket type ultrasound probe interfaces 124 may allow a socket type ultrasound probe to connect to an ultrasound board 232 included in ultrasound system 100. For example, an ultrasound probe connected to socket type ultrasound probe interface 124 may be connected to ultrasound board 232 via transducer/probe socket interface 238. In some embodiments, socket type ultrasound probe interface 124 allows communication between components of portable ultrasound system 100 and other components included in or connected with portable ultrasound system 100. For example, control signals may be provided to an ultrasound probe (e.g., controlling the ultrasound emissions of the probe) and data may be received by ultrasound system 100 from the probe (e.g., imaging data).

In various embodiments, various ultrasound imaging systems may be provided with some or all of the features of the portable ultrasound system illustrated in FIGS. 1A-1B and 2. In various embodiments, various ultrasound imaging systems may be provided as a portable ultrasound system, a portable ultrasound transducer, a hand-held ultrasound device, a cart-based ultrasound system, an ultrasound system integrated into other diagnostic systems, etc.

### B. Systems and Methods for Multi-Resolution Discriminant Analysis of Ultrasound Data

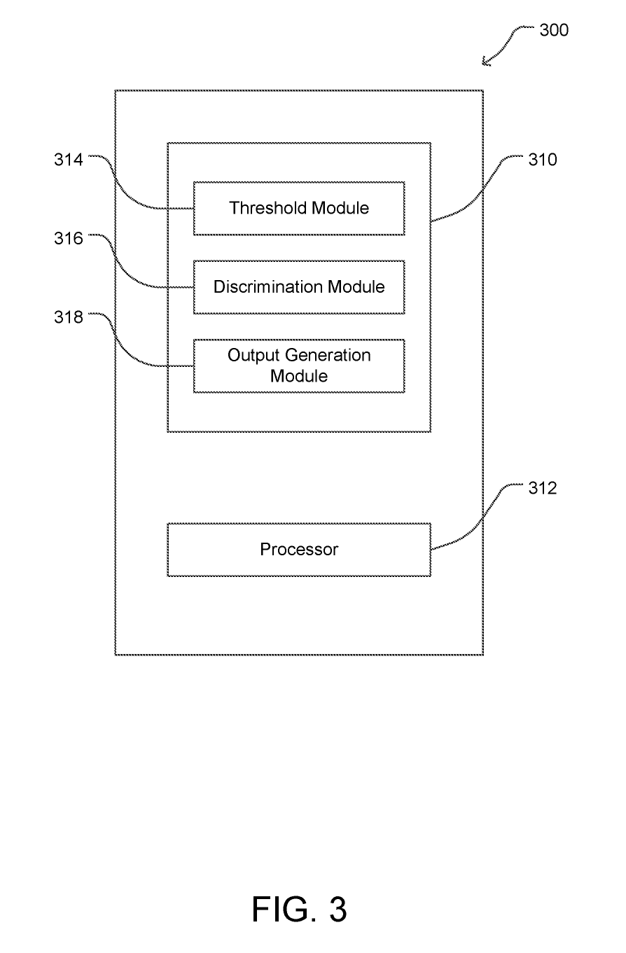

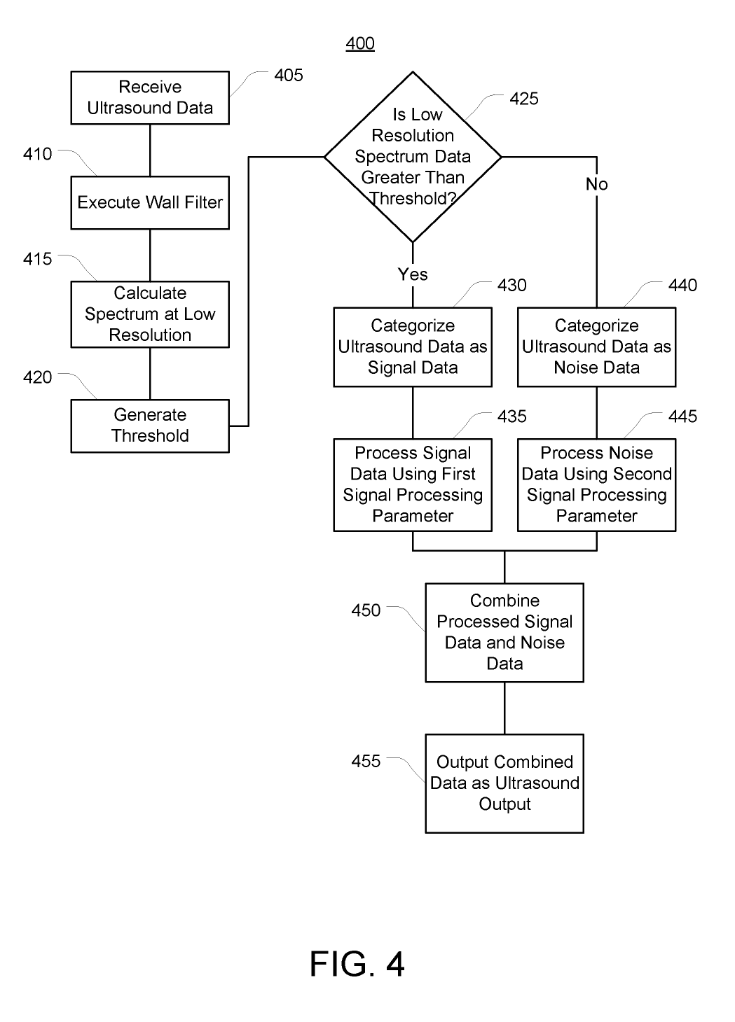

Referring now to FIG. 3, an embodiment of a processing circuit 300 of an ultrasound system (e.g., ultrasound system 100) is illustrated. The processing circuit 300 includes a memory 310 and a processor 312. The processing circuit 300 can be similar to and perform similar functions as the processing circuit 210 described herein with reference to FIG. 2. For example, the memory 310 can be similar to the memory 212, and the processor 312 can be similar to the processor 214. As described herein with reference to FIG. 3, the processing circuit 300 (and particularly, memory 310 thereof) can include various electronic modules (e.g., circuits, software engines, etc.), configured to execute various functions performed by an ultrasound system; in various embodiments, the processing circuit 300 can be organized in various ways for determining how functions are executed. The modules can be configured to share responsibilities by sending instructions to each other to execute algorithms and other functions, and receiving outputs generated by the module receiving the instructions. While FIG. 3 (and FIG. 4) illustrate an example arrangement of modules of the memory 310 and processes executed by the modules, it will be appreciated that the sequence of process execution may be various according to various implementations; for example, the threshold module 314 or discrimination module 316 can be executed before or after gain processing or dynamic range processing is executed.

In some embodiments, the processing circuit 300 is configured to execute morphological or spatial processing of ultrasound information, such as ultrasound data samples or ultrasound images. The processing circuit 300 can receive ultrasound data samples from an ultrasound transducer (e.g., an ultrasound transducer similar or identical to ultrasound transducer assembly 102). The ultrasound data sample can correspond to or represent ultrasound information such as features of blood flow or vasculature of the patient. The ultrasound data sample can be raw data from the ultrasound transducer. For example, the ultrasound data sample can be an analog radio frequency signal outputted by the ultrasound transducer, or a digital data signal resulting from processing of the analog radio frequency signal by an analog-to-digital converter. The ultrasound data sample can represent a velocity of blood at a single point or within a region in space in the patient. The ultrasound data sample can represent a vascular feature of the patient, such as a wall of an artery or a vein.

The ultrasound data sample can correspond to individual points of ultrasound information (e.g., a single point corresponding to amplitude, frequency, time, and/or position information; a single point corresponding to a velocity and time pair), or can be organized into segments corresponding to durations of time, such as durations of time corresponding to a heart cycle of a patient (e.g., sequences of points corresponding amplitude, frequency, time, and/or position information; sequences of points corresponding to velocities paired with times of a heart cycle of a patient). For example, an ultrasound data sample can include a sequence of data point pairs (e.g., raw data) of [frequency, time] corresponding to a heart cycle; or, if a Doppler equation algorithm has been executed to process the raw data, the ultrasound data sample can include a sequence of data point pairs of [velocity, time] corresponding to a heart cycle, or any other sequence of data point pairs corresponding to a Doppler spectrum based on the ultrasound information. The processing circuit 300 may be configured to execute a Doppler equation algorithm to determine velocity information (e.g., velocity as a function of time at a particular position).

The processing circuit 300 can be configured to pre-process the information for the threshold module 314. For example, the processing circuit 300 can execute at least one of a spatial filter algorithm or an edge enhancement algorithm on the ultrasound information, such as to enhance a portion of the ultrasound information corresponding to blood flow. The at least one of the spatial filter algorithm or edge enhancement algorithm can be executed based on brightness information, a signal-to-noise ratio, an identification of signal data, or other factors.

The processing circuit 300 can calculate a plurality of first spectra for a first subset of ultrasound data samples received from the ultrasound transducer. The first subset may be at a low resolution. For example, whereas ultrasound data sampled from raw ultrasound data may typically be sampled to have 64 or 128 samples in a given period of time, the first subset of ultrasound data samples may have 8 or 16 samples in the given period of time.

The processing circuit 300 includes a threshold module 314. The threshold module 314 is configured to generate a threshold based on the plurality of first spectra. In some embodiments, the threshold module 314 generates the threshold as a function of a mean and a standard deviation of the plurality of first spectra, such as shown in Equation 1 below:

Threshold(f)=(μ(f)+k*σ(f))

where f is the frequency of the spectrum, μ is the mean of the spectrum, k is a coefficient which can be adaptively adjusted, and σ is the standard deviation of the spectrum. In some embodiments, the threshold module 314 determines the threshold using fuzzy logic, such as by segmenting signal information from noise information based on a fuzzy logic threshold. The threshold module 314 can determine the threshold using a pattern classification algorithm, including at least one of Fischer's discrimination methods, Voronoi regions, clustering methods, or principle-component analysis. The threshold module 314 can determine the threshold using an image segmentation method (e.g., by segmenting the ultrasound data into segments corresponding to expected regions of the underlying anatomy, and generating the threshold based on the segments). The image segmentation method may include morphological processing. The image segmentation method may include wavelet filtering or Gabor filtering.

In some embodiments, the threshold module 314 executes a static noise reduction algorithm to generate the threshold. The static noise reduction algorithm may be executed based on a predetermined expected characteristic of the noise, such as by using parameters such as several pulse repetition frequencies, probes, or depths. The static noise reduction algorithm may be executed by adapting and/or updating the predetermined expected characteristic based on plurality of use cases, allowing for learning of threshold values to better predict threshold values for future use cases.

In some embodiments, the threshold module 314 adaptively updates the threshold. For example, the threshold module 314 can execute a feedback loop using a pre-defined threshold or a prior threshold to adaptively update the threshold. The threshold module 314 can adaptively update the threshold to reduce the impact of transient effects such as spike noise and/or gaps.

In an embodiment, the threshold module 314 adaptively updates the threshold by: computing a tentative threshold in accordance with Equation 1; comparing a pixel (of the ultrasound data) to the tentative threshold; defining the pixel as a tentative signal pixel if the pixel is greater than the tentative threshold; defining the pixel as a tentative noise pixel if the pixel is less than or equal to the tentative threshold; comparing at least one of a mean or a standard deviation of the tentative noise pixels to a corresponding at least one of a prior (e.g., pre-defined or from a previous threshold calculation) mean or prior standard deviation to determine a tentative difference, comparing the tentative difference to a tentative difference threshold; and outputting the tentative threshold as the threshold to be used by categorization module 316 if the tentative difference is less than the tentative difference threshold, otherwise recalculating the tentative threshold using a lesser k value. As such, the threshold module 315 can adapt the threshold by decreasing the threshold so that excess noise data is not inadvertently being commingled with signal data, and/or so that the threshold is less susceptible to transient changes in the ultrasound data.

The threshold module 314 can also adaptively update the threshold using multi-stage processing. In an embodiment, the threshold module 314 adaptively updates the threshold by executing a first processing stage and a plurality of subsequent processing stages. The first processing stage includes: calculating a first threshold in accordance with Equation 1; categorizing pixels less than the first threshold as tentative noise pixels, otherwise as tentative signal pixels; executing smoothing on at least one of the tentative signal pixels or the tentative noise pixels; and executing a histogram analysis on the tentative noise pixels to refine the tentative noise pixels (e.g., to identify outlier noise pixels which should be recategorized as tentative signal pixels). Each subsequent processing stage includes: calculating a subsequent threshold as a mean of the tentative noise pixels multiplied by the factor k times a standard deviation of the tentative noise pixels; categorizing pixels less than the subsequent threshold as tentative noise pixels, otherwise as tentative signal pixels; executing smoothing on at least one of the tentative signal pixels or the tentative noise pixels; and executing a histogram analysis on the tentative noise pixels to refine the tentative noise pixels. The number of subsequent processing stages executed may be a predetermined number. Alternatively, subsequent processing stages may be executed until a change in the threshold between stages is less than a change threshold, or the number of pixels recategorized from noise to signal is less than a recategorization threshold. It will be appreciated that the value used for the factor k may be refined as well based on a desired computational speed for the threshold calculation and/or a desired signal to noise ratio.

The processing circuit 300 also includes a discrimination module 316. The discrimination module 316 is configured to categorize ultrasound data samples using the threshold determined by the threshold module 314. In some embodiments, the discrimination module 316 compares first spectra to the threshold. Responsive to determining that the first spectra are greater than the threshold, the discrimination module 316 categorizes first spectra as signal data (e.g., flow data). Responsive to determining that the first spectra are less than equal to the threshold, the discrimination module 316 categorizes the first spectra as noise data.

In some embodiments, the discrimination module 316 executes the comparison after executing a Fast Fourier Transform (FFT) on the ultrasound data samples. For example, as shown in Equation 2 below, the discrimination module 316 can calculate the FFT of ultrasound data samples, and compare the result to the threshold for a corresponding frequency:

$$output ( t ) = \{ \begin{matrix}flow ( t ) , & FFT ( input ( t ) ) > threshold ( f ) \\ noise ( t ) , & FFT ( input ( t ) ) \leq threshold ( f )\end{matrix} ,$$

As noted above, the first spectra are generated by sampling ultrasound data at a lesser rate than for typical ultrasound data processing operations, such as by sampling 8 or 16 points over a selected period of time, rather than 64 or 128. In some embodiments, categorizing the first spectra as signal data or noise data includes categorizing other ultrasound data samples from the selected period of time as signal data or noise data. For example, if a selected period of time includes 8 ultrasound data samples selected as the first spectra and 64 total ultrasound data samples, the discrimination module 316 can categorize all 64 ultrasound data samples as signal data or noise data after receiving the threshold from the threshold module 314. In some embodiments, the discrimination module 316 executes spectrum computation on the remaining ultrasound data samples in the selected period of time after executing the threshold module 314. It will be appreciated that because the threshold module 314 calculates the threshold using a relatively small number of ultrasound data samples, the computational burden for distinguishing all ultrasound data samples as either signal data or noise data before further processing may not significantly affect latency of the eventual ultrasound output from the processing circuit 300.

By distinguishing signal data ultrasound data samples from noise data ultrasound data samples, the processing circuit 300 can more effectively perform signal processing operations for improving the appearance and/or sound of the ultrasound output. The processing circuit 300 can increase the signal-to-noise ratio of the ultrasound output relative to existing systems that process signal data and noise data together. The processing circuit 300 can reduce gaps more effectively.

The processing circuit 300 can process the signal data using a first signal processing parameter, and process the noise data using a second signal processing parameter different than the first signal processing parameter. The first and second signal processing parameters may be used for performing the same type of signal processing operation (e.g., applying gain or scaling). The first and second signal processing parameters may be used for performing different signal processing operations. For example, if gain is only applied to signal data but not noise data, it will be appreciated that the first signal processing parameter will have a first value to apply gain (e.g., a value greater than 1), while the second signal processing parameter will have a second value different from the first signal processing parameter (e.g., a value such that no gain is applied, such as 1, or a flag or other indication that gain processing should be skipped for the noise data). The processing circuit 300 can amplify the signal data and/or suppress the noise data to increase the signal-to-noise ratio.

The processing circuit 300 can separately execute pulse wave processing steps on the signal data and on the noise data. This may include executing spectrum computation at a desired spectral resolution. For example, there may be a computational advantage to computing spectra for the noise data at a lower spectral resolution than for signal data.

In some embodiments, the first and second signal processing parameters are gain parameters. For example, the processing circuit 300 can suppress noise by applying a different gain or scaling to the noise data than to the signal data by setting the first signal processing parameter to be greater than the second signal processing parameter.

The first and second signal processing parameters can be smoothing parameters. The processing circuit 300 can thus differentiate noise data from signal data by making the second signal processing parameter different from the first signal processing parameter.

Similarly, the processing circuit 300 can execute at least one of amplification, filtering, or edge processing to differentiate signal data from noise data by making the second signal processing parameter different from the first signal processing parameter. In some embodiments, the at least one of amplification, filtering, or edge processing is perform on the signal data and not on the noise data.

The first and second signal processing parameters can be gap fill parameters. The processing circuit 300 can identify the gap based on regions where signal data is absent or at a relatively low magnitude, but would be expected to be present based on prior data and/or signal data in neighboring regions. For the noise data, the processing circuit 300 can calculate random noise, and add the random noise to the gap to fill the gap in the noise data. In some embodiments, the random noise is calculated from a static template. The random noise may also be calculated dynamically, such as based on characteristics of the noise data. In some embodiments, the processing circuit 300 executes signal persistence using the signal data to fill gaps. For example, the processing circuit 300 can combine prior signal data with current signal data (e.g., using a combination factor which may be a function of time elapsed since the prior signal data was received) to persist the prior signal data.

For the signal data, the processing circuit 300 can execute smoothing using prior data to fill the gap in the signal data. In some embodiments, the signal data used for gap filling is obtained from a prior waveform trace (e.g., a prior duration of time used to generate ultrasound data samples). The processing circuit 300 can identify the prior waveform trace by executing at least one of signal matching or template matching, where the template represents expected characteristics of the prior waveform trace, such as amplitude at selected frequencies. In some embodiments, the processing circuit 300 can estimate at least one of a spatial or temporal location of a heart beat to predict the signal data, and use the predicted signal to fill in the gap.

The processing circuit 300 includes an output generation module 318. The output generation module 318 receives the processed signal data and noise data, and combines the signal data and noise data.

The output generation module 318 can execute various combination algorithms on the signal data and noise data, such as linear combinations or non-linear combinations. The processing circuit 300 can execute the threshold module 314, discrimination module 316, and cause the output generation module 318 to combine the processed signal data and noise data, at any stage during the ultrasound data processing. For example, one or more of these processes can be executed before or after wall filtering, gap filling, spectrum computation, log compression, gain application, dynamic range application, smoothing, or baseline generation for ultrasound video. Similarly, one or more of these processes can be executed before or after wall filtering, gap filling, I/Q signal to left/right signal conversion, upsampling (e.g., upsampling the ultrasound data samples to an audio output frequency such as 44.1 kHz), filtering, digital to analog conversion, amplification, Doppler audio processing, Hilbert filtering, or volume control. The output generation module 318 can combine the signal data and noise data based on at least one of a spatial factor (e.g., depth in the ultrasound image), a time factor (e.g., pulse repetition frequency), or a flow feature (e.g., mean flow velocity, maximum flow velocity). The combination can change as a function of time as the blood flow changes.

The output generation module 318 generates ultrasound output including at least one of an ultrasound image or ultrasound audio using the combined signal data and noise data. The output generation module can modify the ultrasound image by changing parameters of the image such as brightness or color values associated with spatial positions (e.g., pixels) of the binary image.

The image modification module 318 can execute a wall filter configured to identify and remove low-frequency components in ultrasound information detected by the ultrasound transducer assembly 102, such as by applying a high pass filter to the ultrasound information. The high pass filter can be calibrated based on stored information regarding typical frequencies detected for blood flow, as compared to typical frequencies detected for blood vessel walls. The high pass filter can be calibrated dynamically and/or in response to user input, such as user input indicating feedback from a user describing whether a displayed spectrum of ultrasound data includes information representative of blood vessel walls. Based on determining that a vascular feature corresponds to an arterial region or a venous region, the image modification module 318 can recalibrate the wall filter (e.g., modify a filter frequency threshold) to more accurately differentiate blood flow from a vessel wall associated with the vascular feature. In some embodiments, the image modification module 318 executes the wall filter prior to threshold determination by the threshold module 314.

In some embodiments, the image modification module 318 modifies the wall filter based on the signal data and noise data outputted by the categorization module 316. The image modification module 318 can calculate a signal to noise ratio of the signal data and noise data, and compare the signal to noise ratio to a signal to noise ratio threshold, and modify the wall filter if the signal to noise ratio is less than the signal to noise ratio threshold. For example, if the signal to noise ratio is less than the signal to noise ratio threshold, it may be likely that excess noise is being detected from wall data, such that a frequency used to filter out the wall data should be increased.

In some embodiments, the image modification module 318 modifies generation of the ultrasound output based on a signal-to-noise-ratio of the signal data and the noise data categorized by the categorization module 316 based on the threshold generated by the threshold module 314. The image modification module 318 can compare the signal-to-noise ratio to a signal-to-noise ratio threshold. If the signal-to-noise ratio is greater than the signal-to-noise ratio threshold, the image modification module 318 can execute at least one of (1) processing the signal data with a decreased hamming window, which can improve spectral resolution, or (2) increasing the dynamic range of the ultrasound output. If the signal-to-noise ratio is less than or equal to the signal-to-noise ratio threshold, then the output generation module 318 can execute at least one of (1) processing the signal data with an increased hamming window, which can improve the signal-to-noise ratio; (2) increase smoothing to improve the signal-to-noise ratio; or (3) decrease the dynamic range of the ultrasound output.

The output generation module 318 can be configured to modify the image based on user input. For example, the output generation module 318 can receive user input indicating instructions to modify at least one of a gain or a dynamic range of the displayed image. The output generation module 318 can modify a brightness of pixels of the image for display based on the user input.

Referring now to FIG. 4, a method 400 for adaptive enhancement of vascular imaging is illustrated. The method 400 can be implemented by an ultrasound system, such as ultrasound system 100, an ultrasound system including processing circuit 300, etc. The method 400 can be performed for displaying an ultrasound spectrum or image, or outputting ultrasound audio, to a user performing an ultrasound diagnostic procedure.

At 405, ultrasound data is received. For example, ultrasound data from an ultrasound transducer probe can be positioned adjacent to the patient to detect ultrasound information from the patient. The ultrasound transducer probe can output the ultrasound data as frequency information. In some embodiments, the ultrasound transducer probe can be configured to process the frequency information into velocity information as a function of time, and output the ultrasound data as the velocity information as a function of time.

At 410, a wall filter is executed. The wall filter can be executed by applying a high pass filter to the ultrasound data.

At 415, a spectrum is calculated using the ultrasound data at a low-resolution. For example, if ultrasound data is typically processed by extracting 64 or 128 ultrasound data samples from a selected period of time, the low-resolution calculation may be based on 8 or 16 ultrasound data samples from the selected period of time.

At 420, a threshold is generated using the low-resolution spectrum. The threshold may be generated as a function of a mean and a standard deviation of the plurality of the low-resolution spectrum. The threshold may be generated by updating a previous threshold. The threshold may be adaptively updated using a feedback loop comparing the threshold to a pre-defined threshold or a previous threshold. The threshold may be adaptively updated using multi-stage processing.

At 425, the low-resolution spectrum is compared to the threshold. If the low-resolution spectrum is greater than the threshold, then at 430, the low-resolution spectrum is categorized as signal data. Categorizing the low-resolution spectrum as signal data may include categorizing other ultrasound data samples and/or ultrasound spectra from the associated duration of time as signal data.

At 435, the signal data is processed using a first signal processing parameter. Processing the signal data may include calculating spectra from the ultrasound data at a desired resolution (if not already done so). The first signal processing parameter may be a gain parameter a scaling parameter, a wall filter parameter, a gap fill parameter, a smoothing parameter, an amplification parameter, a filtering parameter, or an edge processing parameter.

If the low-resolution spectrum is less than or equal to the threshold, then at 440, the ultrasound data is categorized as noise data. Categorizing the low-resolution spectrum as noise data may include categorizing other ultrasound data samples and/or ultrasound spectra from the associated duration of time as signal data.

At 445, the noise data is processed using a second signal processing parameter different from the first signal processing parameter. The second signal processing parameter may be of a same type as the first signal processing parameter (e.g., both parameters are gain parameters having different values) or of a different type (e.g., gain is only applied to signal data, so the second processing parameter may have a value of 0 or 1 for applying gain, or may be a flag indicating that gain should not be applied to the noise data).

At 450, the processed signal data and processed noise data are combined into ultrasound output. The processed signal data and processed noise data may be combined using a linear combination or a non-linear combination. The processed signal data and processed noise data may be combined at various stages in a signal processing pathway from receiving raw data to outputting ultrasound output.

At 455, the ultrasound output is outputted. The ultrasound output may be outputted as ultrasound image(s) (e.g., in various ultrasound image modes such as B-mode, duplex, triplex). The ultrasound output may be outputted as audio.

In various embodiments, ultrasound systems operated in accordance with the systems and method described herein can improve upon existing ultrasound systems by more effectively discriminating signal data from noise data, such as in pulse wave Doppler operation, at multiple spectral resolutions. The signal data may be discriminated from noise data in either the time domain or frequency domain. The discrimination can be used to suppress noise while enhancing signal information. The discrimination can be used to more effectively fill gaps in duplex and triplex mode. These improvements may be realized in both visual image output as well as audio output.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures may show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system, comprising:
an ultrasound transducer configured to detect ultrasound information regarding a patient and output the ultrasound information as ultrasound data samples;
a processing circuit configured to:
receive a plurality of ultrasound data samples from the ultrasound transducer;
calculate a plurality of first spectra for a first subset of the received ultrasound data samples;
generate a threshold based on the plurality of first spectra;
compare each of the plurality of first spectra to the threshold;
categorize first spectra greater than the threshold as signal data;
categorize first spectra less than or equal to the threshold as noise data;
process the signal data using a first signal processing parameter;
process the noise data using a second signal processing parameter different from the first signal processing parameter; and
combine the processed signal data and noise data into an ultrasound output; and
an output device including at least one of a display configured to display the ultrasound output as an ultrasound image or an audio output device configured to output the ultrasound output as ultrasound audio.

2. The system of claim 1, wherein the processing circuit is further configured to generate the threshold as a function of a mean of the plurality of first spectra and a standard deviation of the plurality of first spectra.

3. The system of claim 2, wherein the function is the mean summed with a factor k multiplied by the standard deviation, and the processing circuit is further configured to adaptively update the threshold by:
calculating a difference between the threshold and at least one of (1) a previously generated threshold or (2) a pre-defined threshold;
comparing the difference to a difference threshold;
if the difference is greater than the difference threshold, decreasing the factor k; and
if the difference is less than or equal to the difference threshold, outputting the adaptively updated threshold as the threshold.

4. The system of claim 2, wherein the processing circuit is further configured to adaptively update the threshold by executing a series of smoothing operations.

5. The system of claim 1, wherein the processing circuit is further configured to execute a wall filter algorithm using the plurality of ultrasound samples prior to calculating the plurality of first spectra.

6. The system of claim 5, wherein the processing circuit is further configured to calculate a signal to noise ratio of the signal data and noise data, compare the signal to noise ratio to a signal to noise ratio threshold, and modify the wall filter algorithm if the signal to noise ratio is less than the signal to noise ratio threshold.

7. The system of claim 1, wherein the processing circuit is further configured to calculate a signal to noise ratio of the signal data and noise data, compare the signal to noise ratio to a signal to noise ratio threshold, and if the signal to noise ratio is less than the signal to noise ratio threshold, execute at least one of (1) increasing the first signal processing parameter, wherein the first signal processing parameter is a hamming window, (2) increasing the first signal processing parameter, wherein the first signal processing parameter is a smooth parameter, or (3) causing the display to display the ultrasound output with a reduced dynamic range.

8. The system of claim 1, wherein the first signal processing parameter and second signal processing parameter include at least one of a gain parameter or a scaling parameter.

9. The system of claim 1, wherein the processing circuit is configured to compare the first spectra to the threshold in a time domain or in a frequency domain.

10. The system of claim 1, wherein the processing circuit is further configured to process the signal data by applying spectral smoothing to the signal data and process the noise data by applying random noise filling to the noise data.

11. The system of claim 1, wherein the processing circuit is configured to generate the threshold by executing at least one of a pattern discrimination, image segmentation, or static noise reduction.

12. The system of claim 1, wherein the processing circuit is configured to combine the signal data and noise data as a function of at least one of a depth in the ultrasound image, a pulse repetition frequency, or a velocity of the signal data.

13. The system of claim 1, wherein the first signal processing parameter and second signal processing parameter are associated with a gap filling process executed by the processing circuit.

14. A method, comprising:
receiving a plurality of ultrasound data samples associated with ultrasound information regarding a patient;
calculating a plurality of first spectra for a first subset of the received ultrasound data samples;
generating a threshold based on the plurality of first spectra;
comparing each of the plurality of first spectra to the threshold;
categorizing first spectra greater than the threshold as signal data;
categorizing first spectra less than or equal to the threshold as noise data;
processing the signal data using a first signal processing parameter;
processing the noise data using a second signal processing parameter different from the first signal processing parameter;
combining the processing signal data and noise data into an ultrasound output; and
at least one of displaying the ultrasound output as an ultrasound image or outputting the ultrasound output as ultrasound audio.

15. The method of claim 14, further comprising adaptively updating the threshold by:
generating the threshold as a mean of the plurality of first spectra summed with a factor k multiplied by the standard deviation;
calculating a difference between the threshold and at least one of (1) a previously generated threshold or (2) a pre-defined threshold;
comparing the difference to a difference threshold;
if the difference is greater than the difference threshold, decreasing the factor k; and
if the difference is less than or equal to the difference threshold, outputting the adaptively updated threshold as the threshold.

16. The method of claim 14, further comprising adaptively update the threshold by executing a series of smoothing operations.

17. The method of claim 14, further comprising calculating a signal to noise ratio of the signal data and noise data, comparing the signal to noise ratio to a signal to noise ratio threshold, and if the signal to noise ratio is less than the signal to noise ratio threshold, at least one of (1) increasing the first signal processing parameter, wherein the first signal processing parameter is a hamming window, (2) increasing the first signal processing parameter, wherein the first signal processing parameter is a smooth parameter, or (3) causing the display to display the ultrasound output with a reduced dynamic range.

18. A portable ultrasound device, comprising:
a processing circuit configured to:
calculate a plurality of first spectra for a first subset of the received ultrasound data samples;
generate a threshold based on the plurality of first spectra;
compare each of the plurality of first spectra to the threshold;
categorize first spectra greater than the threshold as signal data;
categorize first spectra less than or equal to the threshold as noise data;
process the signal data using a first signal processing parameter;
process the noise data using a second signal processing parameter different from the first signal processing parameter; and
combine the processed signal data and noise data into an ultrasound output.

19. The portable ultrasound device of claim 18, wherein the processing circuit is further configured to generate the threshold as a function of a mean of the plurality of first spectra and a standard deviation of the plurality of first spectra.

20. The portable ultrasound device of claim 18, wherein the processing circuit is further configured to calculate a signal to noise ratio of the signal data and noise data, compare the signal to noise ratio to a signal to noise ratio threshold, and if the signal to noise ratio is less than the signal to noise ratio threshold, execute at least one of (1) increasing the first signal processing parameter, wherein the first signal processing parameter is a hamming window, (2) increasing the first signal processing parameter, wherein the first signal processing parameter is a smooth parameter, or (3) causing the display to display the ultrasound output with a reduced dynamic range.
